(19) United States
(12) Patent Application Publication
(10) Pub. No.: US 2003/0147865 A1
(43) Pub. Date: Aug. 7, 2003

(54) Cell therapy using immunoregulatory T-cells

(76) Inventors: Benoit Salomon, Paris (FR); Jose Cohen, Paris (FR); David Klatzmann, Paris (FR)

Correspondence Address: NIXON & VANDERHYE P.C., 8th Floor, 1100 North Glebe Road, Arlington, VA, 22201, US

(21) Appl. No.: 10/067,503

(22) Filed: Feb. 7, 2002

(51) Int. Cl. A61K 48/00

(52) U.S. Cl. 424/93.21

(57) ABSTRACT

The present invention relates to the fields of biology, genetics and medicine. The invention discloses methods and compositions for treating various diseases using populations or compositions of immunoregulatory T cells. The invention discloses that regulatory T cells may be produced and used to control in vivo various pathological conditions, including diseases associated with abnormal T cell activity. The invention relates to the manufacture of such regulatory T cell compositions as well as to their uses for cell therapy treatment. The invention is particularly suited for controlling graft versus host disease in subjects undergoing transplantation (e.g., bone marrow transplantation).

The present invention relates to the fields of biology, genetics and medicine. The invention discloses methods and compositions for treating various diseases using populations or compositions of immunoregulatory T cells. The invention discloses that regulatory T cells may be produced and used to control in vivo various pathological conditions, including diseases associated with abnormal T cell activity. The invention relates to the manufacture of such regulatory T cell compositions as well as to their uses for cell therapy treatment. The invention is particularly suited for controlling graft versus host disease in subjects undergoing transplantation (e.g., bone marrow transplantation).

Graft-versus-host disease, the life-threatening and frequent complication of allogeneic hematopoietic stem cell transplantation [Thomas, 1975 #1], is due to mature donor T-cells present in the transplant. However, removal of these T-cells before grafting leads to graft failure, prolonged immunosuppression and leukemia relapse [Marmont, 1991 #3; Mackall, 1997 #2]. To date, standard immunosuppressive treatments of graft-versus-host disease are only partially efficient [Storb, 1989 #32; Socie, 1998 #33], emphasizing the need to develop innovative therapeutic strategies.

The present application proposes such a novel therapeutic approach to the treatment and control of Graft-versus-host disease (GVHD) and other immune diseases in a subject. The present application unexpectedly shows that the few CD4⁺CD25⁺ T-cells naturally present in hematopoietic stem cell transplants regulate GVHD, since their removal from the transplant dramatically accelerates graft-versus-host disease. Furthermore, the present application shows that the addition of freshly isolated CD4⁺CD25⁺ T-cells at time of grafting significantly delays or even prevents graft-versus-host disease. Moreover, the present application also demonstrates that ex vivo expanded CD4⁺CD25⁺ T-cells, which have been activated by recipient cells, can also control graft-versus-host disease. Thus, CD4⁺CD25⁺ regulatory T-cells represent a new accessible therapeutic approach for controlling immune dysfunctions in a subject, particularly graft-versus-host disease in allogeneic hematopoietic stem cell transplantation.

An object of the present application thus relates to the use of (human) immunoregulatory T cells for the manufacture of a composition for cell therapy treatment of an immune disease in a subject.

An other object of this application is a method of treating an immune disease in a subject, comprising administering to a subject in need thereof an effective amount of (human) immunoregulatory T cells, particularly an amount of immunoregulatory T cells effective at suppressing a pathological immune response.

As will be discussed below, the immunoregulatory T cells may be freshly isolated cells or ex vivo expanded immunoregulatory T cells. Furthermore, these cells may be genetically modified to express desired biological products. Where ex vivo (or in vitro) expanded immunoregulatory T cells are used, they may be expanded (and activated) by different methods using either non-specific or antigen-specific stimulation, depending on the disease or condition to be treated. Depending on the pathology, the immunoregulatory T cells used in the present invention are autologous, i.e., they originate from the subject to be treated, or allogeneic towards the patient (e.g., they originate from a donor subject, typically from the subject from which the organ or transplanted material originates).

The invention is suited for treating various diseases caused by pathological T cells, including graft versus host disease, autoimmune diseases, graft rejection, allergies, immunopathologies mediated by viruses, etc. It is particularly suited for the treatment of graft versus host disease in a subject undergoing allogeneic organ transplantation, for example allogeneic bone marrow or hematopoietic stem cell transplantation. It is also particularly suited for treating diabetes or allergies.

In this regard, a particular object of the present invention resides in the use of freshly isolated or ex vivo expanded human immunoregulatory T cells for the manufacture of a composition for the treatment of graft versus host disease in a subject undergoing allogeneic bone marrow (or HSC) transplantation, as well as a corresponding method of treatment. In said particular use or method, the immunoregulatory T cells may be administered to the subject together with the bone marrow transplant, before the bone marrow transplant or after the bone marrow transplant.

An other particular object of the present invention resides in the use of freshly isolated or ex vivo expanded human immunoregulatory T cells for the manufacture of a composition for the treatment of an auto-immune disease in a subject, as well as a corresponding method of treatment. In said particular use or method, the immunoregulatory T cells may be expanded using auto-antigens specific for said disease or condition to be treated, in order to increase the therapeutic efficacy.

An other particular object of the present invention resides in the use of freshly isolated or ex vivo expanded human immunoregulatory T cells for the manufacture of a composition for the treatment of allergy in a subject, as well as a corresponding method of treatment. In said particular use or method, the immunoregulatory T cells may be expanded using allergens, in order to increase the therapeutic efficacy.

A further object of this invention is a composition comprising (i) freshly isolated or ex vivo expanded human immunoregulatory T cells and (ii) donor effector T cells, for combined, separate or sequential use. The composition may further comprise (iii) hematopoietic stem cells.

An other particular object of this application is a composition comprising genetically modified freshly isolated or ex vivo expanded (human) immunoregulatory T cells and a pharmaceutically acceptable medium or vehicle. In a preferred embodiment, the genetically modified immunoregulatory T cells comprise a recombinant nucleic acid molecule encoding a product with conditional toxicity to said cells, such as a thymidine kinase. In an other preferred embodiment, the genetically modified immunoregulatory T cells comprise a recombinant nucleic acid molecule encoding a T cell receptor or a sub-unit or functional equivalent thereof.

The invention also relates to methods of producing human immunoregulatory T cells, particularly expanded and/or genetically modified human immunoregulatory T cells. Expansion of immunoregulatory T cells is preferably performed by culturing a cell population comprising immunoregulatory T cells in the presence of a cytokine and a T cell and/or antigen-specific stimulating agent or condition, such as antigens, cells, antibodies, lectins, etc. Genetic modification is preferably accomplished using a recombinant virus carrying a desired recombinant nucleic acid molecule, typically a retrovirus. separated on another column. Purification is generally performed in phosphate buffer saline, although other suitable medium may be used. The cells may be maintained in any suitable buffer or medium, such as saline solution, buffer, culture media, particularly DMEM, RPMI and the like. They may be frozen or maintained in cold condition. They can be formulated in any appropriate device or apparatus, such as a tube, flask, ampoule, dish, syringe, pouch, etc., preferably in a sterile condition suitable for pharmaceutical use.

The cells used in performing the present invention are thus typically isolated immunoregulatory T cells, i.e., a composition enriched for said cells, preferably a composition comprising at least 30%, preferably at least 50%, even more preferably at least 65% of immunoregulatory T cells. Particularly preferred compositions or cells for use in the present invention comprise at least 75%, preferably at least 80% of immunoregulatory T cells. The compositions may comprise other cell types or T cell subpopulations, without affecting significantly the therapeutic benefit of the present invention. If desired, specific cell types may be depleted from the composition using particular antibodies or markers. For instance, effector T cells specific for autoantigens may be eliminated by depletion using such antigens (or fragments thereof) coated on a support.

The cells may be cultured in any appropriate media, as disclosed above. For performing the present invention, it is possible to use immunoregulatory T cells freshly isolated from a biological fluid, or immunoregulatory T cells that have been expanded ex vivo or in vitro. In this regard, ex vivo or in vitro expansion is preferably obtained by culturing the cells in the presence of a stimulating agent and a cytokine, for a period of time sufficient to expand (amplify, multiply) the cell population, essentially without altering their CD4+CD25+phenotype. The stimulating agent may be an antigen-presenting cell (“APC”), i.e., any cell presenting antigens or any cell supporting activation of immunoregulatory T cells.

Preferably, the APCs are irradiated prior to their use in order to avoid their expansion. The APCs may be cells isolated from the donor, or allogeneic cells. They may be selected to produce activated immunoregulatory T cells having a desired activity profile. Typical examples of such APCs include peripheral blood mononuclear cells, splenocytes, cells from cord blood, tissue or organ samples, etc. Other suitable T cell stimulating agents include MHC polymers, lectins (such as PHA), antibodies (such as anti-CD3 antibodies) or fragments thereof, auto-antigens (including tissues, cells, cell fragments or debris, purified polypeptides or peptides, etc., preferably in combination with antigen-presenting cells), etc. Activation usually requires culture in the presence of a cytokine, typically interleukin-2 or interleukin-15, preferably of human origin. Depending on the disease or condition to be treated, the immunoregulatory T cells can be expanded by different ways, whether antigen-specific or not. In particular, for some indications, high numbers of the whole repertoire of immunoregulatory T cells can be preferably used (e.g., injected). This is specifically indicated for patients that present a global deficit (quantitative or functional) in immunoregulatory T cells, such as in type-i diabetic patients (J Clinical Invest 109:131). In such indications, the cells are preferably expanded for example by autologous APCs and PHA or anti-CD3 antibodies (or any other T-cell activators) in the presence of cytokine(s).

Alternatively more specific expansion can be envisioned, particularly where immunosuppression of specific effector T cells is sought, such as in autoimmune diseases, allergy, graft-rejection, GVHD, etc. In such indications, the cells are preferably expanded in the presence of an antigen-specific activating signal, to favor expansion of immunoregulatory T cells active against particular clones or repertoire of effector T cells. situations, for instance in the treatment of GVHD or other diseases, the immunoregulatory T cells are typically allogeneic, i.e., they originate from a different human being. In these cases, it is preferred to use immunoregulatory T cells that originate from the donor subject (e.g., from the donor subject of effector cells).

In a most preferred embodiment, the immunoregulatory T cells are obtained by a method comprising:

a) providing a biological sample comprising lymphocytes, preferably from the subject to be treated or from a donor subject,

b) isolating immunoregulatory T cells from said sample, preferably by selecting CD25-positive cells,

c) optionally expanding the immunoregulatory T cells by activation in the presence of a stimulating agent and a cytokine,

d) optionally genetically modifying the immunoregulatory T cells by contacting said cells with a recombinant nucleic acid molecule, preferably by virus-mediated gene transfer, and

e) conditioning said cells in the presence of a pharmaceutically acceptable medium or vehicle.

In a typical embodiment, the cells are conditioned in a composition that comprises between about 10E5 and about 10E10 immunoregulatory T cells depending on the disease, more generally between about 10E5 and about 10E9 immunoregulatory T cells. As a general indication, for treating graft versus host disease, it is generally preferred to use the same proportion of immunoregulatory cells as donor T cells administered with the transplant. It should be understood that repeated administrations may be performed.

The invention recognizes and establishes the therapeutic potential of compositions comprising freshly isolated or expanded immunoregulatory T cells. The invention may be used to treat various subjects, typically human patients suffering from or having a risk of developing an immune disease, particularly a disease caused by a pathological T cell response. The treatment may be preventive or curative. It may be combined with other treatments.

Human Immunoregulatory T Cells

Within the context of the present application, immunoregulatory T cells designate a population of T cells that express particular cell surface markers, namely CD4 and CD25 markers. These cells are thus also referred to as CD4⁺CD25⁺ regulatory cells. The immunoregulatory CD4⁺CD25⁺ T cells generally represent 3-10% of the normal T-cell compartment in mice and humans [Sakaguchi, 1995 #19; Levings, 2001 #36]. These cells are characterized by an ability to suppress or downregulate immune reactions mediated by effector T cells, such as effector CD4+ or CD8+ T cells. Immunoregulatory T cells may be obtained from various biological samples containing lymphocytes, such as blood, plasma, lymph node, immune organs, etc. Typically, they are isolated or collected from peripheral blood. They may be isolated by contacting such a biological fluid with specific ligands, such as anti-CD25 antibodies or fragments or derivatives thereof having the same antigen specificity. Such labelled cells may then be separated by various techniques such as affinity chromatography, cell sorting, etc. In a typical embodiment, peripheral blood cells are sequentially incubated with saturating amounts of functionalized (e.g., biotin-labeled) anti-CD25 antibody and with a functionalized (e.g., streptavidin-coated) solid support (such as microbeads). The cells are then purified by recovering the support, e.g., by magnetic cell separation. To increase cell purification, the cells of the positive fraction may be further

In this regard, in the particular case of Graft versus host disease, donor-type immunoregulatory T cells are preferably used and stimulated by antigen presenting cells isolated from the recipient prior to the hematopoietic stem cell transplantation (HSCT). These ex vivo expanded immunoregulatory T cells are then injected to the recipient subject, at the same time as the HSCT or a few days before or after. Injection of immunoregulatory T cells can be repeated after the HSCT.

For treating autoimmune diseases, immunoegulatory T cells are preferably isolated from the patient and stimulated by autologous APCs and auto-antigens from the target tissue, in the presence of cytokine(s). Auto-antigens can be either tissues, cells, cell fragments, purified proteins, peptides, nucleic acids, etc.

For the treatment of allografts or xenografts, immunoegulatory T cells are typically isolated from the patient and stimulated by APCs or tissues from the donor in the presence of cytokine(s). Alternatively, regulatory T cells isolated from the patient may be stimulated by autologous APCs in the presence of tissues, cells, cell fragments, purified proteins or peptides from the donor and cytokine(s).

For treating allergies, immunoegulatory T cells are typically isolated from the patient and stimulated by autologous APCs and allergens in the presence of cytokines. As indicated above, the cytokine is preferably interleukin-2 or interleukin-15.

In a particular embodiment, the immunoregulatory T cells are genetically modified to encode desired expression products, as will be further described below.

As indicated above, for treating various immunopathologies such as for instance organ transplant rejection, auto-immune diseases, allergies, viro-induced pathologies, etc., the immunoregulatory T cells are typically autologous, i.e., they originate from the subject to be treated. It should be understood that syngeneic cells may be used as well. In other

Genetic Modification of Immunoregulatory T Cells

As indicated above, in particular embodiments, the present invention uses genetically modified immunoregulatory T cells. The term “genetically modified” indicates that the cells comprise a nucleic acid molecule not naturally present in non-modified immunoregulatory T cells, or a nucleic acid molecule present in a non-natural state in said immunoregulatory T cells (e.g., amplified). The nucleic acid molecule may have been introduced into said cells or into an ancestor thereof.

A number of approaches can be used to genetically modify immunoregulatory T lymphocytes, such as virus-mediated gene delivery, non-virus-mediated gene delivery, naked DNA, physical treatments, etc. To this end, the nucleic acid is usually incorporated into a vector, such as a recombinant virus, a plasmid, phage, episome, artificial chromosome, etc.

In a particular embodiment of the invention, the immunoregulatory T lymphocytes are genetically modified using a viral vector (or a recombinant virus). In this embodiment, the heterologous nucleic acid is, for example, introduced into a recombinant virus which is then used to infect immunoregulatory T lymphocytes. Different types of recombinant viruses can be used, in particular recombinant retroviruses or AAV.

In a preferred embodiment, the immunoregulatory T lymphocytes are genetically modified using a recombinant retrovirus. Retroviruses are preferred vectors since retroviral infection results in stable integration into the genome of the cells. This is an important property because lymphocyte expansion, either in vitro or in vivo after injection into the subject, requires that the transgene is maintained stable during segregation in order to be transmitted to each cell division. Examples of retrovirus types which can be used are retroviruses from the oncovirus, lentivirus or spumavirus family. Particular examples of the oncovirus family are slow oncovirus, non oncogene carriers, such as MoMLV, ALV, BLV or MMTV, and fast oncoviruses, such as RSV. Examples from the lentivirus family are HIV, SIV, FIV or CAEV.

Techniques for constructing defective recombinant retroviruses have been widely described in the literature (WO 89/07150, WO 90/02806, and WO 94/19478, the teachings of which are incorporated herein in their entirety by reference). These techniques usually comprise the introduction of a retroviral vector comprising the transgene into an appropriate packaging cell line, followed by a recovery of the viruses produced, said viruses comprising the transgene in their genome.

In a particular embodiment of the invention, a recombinant retrovirus comprising a GALV virus envelope (retrovirus pseudotyped with GALV) is advantageously used. It has been shown that infection of hematopoietic cells by a recombinant retrovirus is more effective when the retroviral envelope is derived from a retrovirus envelope known as the Gibbon Ape Leukemia Virus (GALV) (Movassagh et al., Hum. Gen Ther. 9 (1998) 225, the teachings of which are incorporated herein in their entirety by reference). Using this retroviral envelope, we have shown that it was possible to obtain transduction rates of over 95% in lymphocytes before any selection of transduced cells (unpublished results).

Other particular embodiments use a retrovirus produced in a packaging cell line expressing a truncated pol protein, transient production, retroviruses having a modified tropism, etc.

The immunoregulatory T lymphocytes can be infected with recombinant viruses using various protocols, such as by incubation with a virus supernatant, with purified viruses, by co-culturing the immunoregulatory T lymphocytes with the virus' packaging cells, by Transwell techniques, etc. A particularly effective method has been described by Movassagh et al. (see above), comprising a centrifugation step.

Non-viral techniques include the use of cationic lipids, polymers, peptides, synthetic agents, etc. Alternative methods use gene gun, electrical fields, bombardment, precipitation, etc. In performing the present invention, it is not necessary that all immunoregulatory T cells be genetically modified. It is thus possible to use a population of immunoregulatory T lymphocytes comprising at least 50%, preferably at least 65%, more preferably at least 80% of genetically modified lymphocytes. Higher levels (e.g., up to 100%) can be obtained in vitro or ex vivo; for example using a GALV envelope and/or certain infection conditions (Movassagh et al., above) and/or by selecting the cells which have effectively been genetically modified. In this regard, different selection techniques are available, including the use of antibodies recognizing specific markers on the surface of the modified cells, the use of resistance genes (such as the gene for resistance to neomycin and the drug G418), or the use of compounds which are toxic to cells not expressing the transgene (i.e., thymidine kinase). Selection is preferably carried out using a marker gene expressing a membrane protein. The presence of this protein permits selection using conventional separation techniques such as magnetic bead separation, columns, or flux cytometry.

The nucleic acid used to genetically modify immunoregulatory T cells may encode various biologically active products, including polypeptides (e.g., proteins, peptides, etc.), RNAs, etc. In a particular embodiment, the nucleic acid encode a polypeptide having an immunosuppressive activity. In an other embodiment, the nucleic acid encodes a polypeptide which is toxic or conditionally toxic to the cells. Preferred examples include a thymidine kinase (which confers toxicity in the presence of nucleoside analogs), such as HSV-1 TK, a cytosine desaminase, gprt, etc. An other preferred category of nucleic acids are those encoding a T cell receptor or a sub-unit or functional equivalent thereof. The expression of recombinant TCRs specific for an auto-antigen produces immunoregulatory T cells which can act more specifically on effector T cells that destroy a tissue in a subject. Other types of biologically active molecules include growth factors, lymphokines (including various cytokines that activate immunoregulatory T cells), accessory molecules, antigen-presenting molecules, antigen receptors, etc. In this regard, the nucleic acid may encode “T-bodies”, i.e., hybrid receptors between T cell receptor and an immunoglobulin. Such “T-bodies” allow the targeting of complex antigens, for instance.

The nucleic acid which is introduced into immunoregulatory T cells according to this invention typically comprises, in addition to a coding region, regulatory sequences, such as a promoter and a polyadenylation sequence.

A particular object of this application is a composition comprising genetically modified freshly isolated or ex vivo expanded (human) immunoregulatory T cells and a pharmaceutically acceptable medium or vehicle. In a preferred embodiment, the genetically modified immunoregulatory T cells comprise a recombinant nucleic acid molecule encoding a product with conditional toxicity to said cells, such as a thymidine kinase.

Treatment

The present invention is suited for treating various diseases associated with pathological T cells, as discussed above. The treatment may be preventive or curative. Furthermore, the methods and compositions of this invention may be used in combination with other active agents or principles, such as other cell populations, immunosuppressive drugs or conditions, irradiations, gene therapy products, etc.

The term treatment designates a reduction in the symptoms or causes of a disease, a regression of a disease, a delaying of the development of a disease, an amelioration of the state of patients, a reduction in their suffering, an increase in their life duration, etc.

The invention is particularly suited to delay or prevent GVHD in subjects undergoing allogeneic organ transplantation, particularly bone marrow (or hematopoietic stem cell) transplantation. Graft-versus-host disease, the life-threatening and frequent complication of allogeneic hematopoietic stem cell transplantation, is due to mature donor T-cells present in the transplant. However, removal of these T-cells before grafting leads to graft failure, prolonged immunosuppression and leukemia relapse. The present application unexpectedly shows that the addition of freshly isolated CD4⁺CD25⁺ T-cells at time of grafting significantly delays or even prevents graft-versus-host disease. Moreover, the present application also demonstrates that ex vivo expanded CD4⁺CD25⁺ T-cells, which have been activated by recipient cells, can also control graft-versus-host disease.

The invention is also suited for the treatment of autoimmune diseases (including chronic inflammatory diseases), such as systemic lupus erythematosus, rheumatoid arthritis, polymyositis, multiple sclerosis, diabetes, etc. Autoimmune diseases have a clear immunological component, as shown by various biological and histological investigations. For these diseases, the central element is an unsuitable immune response. Furthermore, in these diseases it is generally possible to identify the auto-antigen and to define the period of time during which the deleterious effector T cells are activated. The present invention can be used to treat, reduce or alleviate such diseases by administering to the subject an effective an amount of immunoregulatory T cells effective to suppress or reduce the activity of such deleterious effector T cells. Repeated administrations may be contemplated, if needed.

The invention is also suited for the treatment of viro-induced immunopathologies. The immune response against infectious agents may have immunopathological consequences which may lead to death. The most common example is that of the response to certain viruses responsible for hepatitis. These viruses replicate in hepatocytes and the destruction of these infected hepatocytes by the immune system results in hepatitis, which is sometimes mortal. The evolution of this chronic hepatitis is accompanied by biological signs indicating a dysimmune response (for example, frequent presence of anti-DNA antibodies or of cryoglobulinemia). The present invention can eliminate, suppress or reduce the active T lymphocytes responsible for the immunopathology, and thus reduce the consequences of viro-induced immunopathologies.

The present invention can also be used for the treatment or the prevention of organ transplant rejection, such as heart, liver, cornea, kidney, lung, pancreas, etc. The conventional treatment for a certain number of organ disorders is, when it becomes necessary, replacement of this organ with a healthy organ originating from a dead donor (or a living donor in certain cases, or even a donor from another species). This is also the case for treating certain insulin-dependent diabetes, through the grafting of insulin-producing cells or organs, such as pancreas or pancreatic islets. While rigorous care is taken in selecting the organ donors with the maximum compatibility vis-a-vis the histocompatibility antigens, apart from transplants between homozygotic twins, the organ transplant always leads to the development of an immune response directed against the antigens specifically expressed by that organ. Despite immunosuppressor treatments carried out, this reaction often results in rejection of the transplanted organ (this is the main cause of failure of allogeneic transplants). Apart from certain super-acute or acute rejections which involve essentially humoral responses, in the majority of cases, organ transplant rejection is essentially mediated by effector T lymphocytes.

In addition, a number of approaches have been developed to deliver biologically active products using allogeneic or xenogenic, modified or non-modified cell transplants (cells from the islets of the pancreas, fibroblasts, etc.). In particular, this has been proposed in diseases as disparate as diabetes, Parkinson's disease, or even in gene therapy in organoids. The principal obstacle to such transplants remains rejection of these allo- or xenogenic cells. To overcome this disadvantage, a very large number of devices have been proposed to separate the grafted cells from the immune system. These systems vary from microencapsulation to insertion of cells in porous materials or semi-permeable materials, etc. Unfortunately, none of those systems has proved to be sufficiently effective to be able to be used clinically.

The present invention now provides a novel approach to the treatment (e.g., the reduction or delay ) of organ rejection using immunoregulatory T cells. Such cells may be prepared from the patient, stimulated with antigens from the donor, and reinfused to the patient, prior to, together with or after organ transplant. Repeated infections may be performed if desired. This approach is particularly suited for treating diabetes, i.e., for reducing, delaying or preventing rejection of transplanted insulin-producing cells, tissues or organs (particularly pancreatic islets. Typically, immunoregulatory T cells are expended and activated by culture in the presence of auto-antigens from the donor tissue. These cells may be produced for instance by culture in the presence of dendritic cells that are autologous with respect to the graft. These expanded and educated immunoregulatory T cells can then be injected to the patient, either before, together and/or after organ transplantation, thereby reducing the destructive activity of effector T cells.

The invention is also suited for the treatment of allergies, which are mediated by immune responses against particular antigens called allergens. By administering to the patients immunoregulatory T cells activated ex vivo with such allergens, it is possible to reduce these deleterious immune responses.

Various administration routes and protocols may be used to perform the present invention. These may be adapted by the skilled person, depending on the pathology to be treated. Generally, systemic or local administration(s) may be envisioned, such as intravenous, intraarterial, intraperitoneal, intramuscular, subcutaneous, etc. The cells may be injected during surgery or by any suitable means, such as using a syringe, for instance. For controlling diseases like GVHD or organ transplant rejection, the cell composition may be administered prior to, during or after bone marrow (or HSC or organ) transplantation. Furthermore, additional administrations may be performed after transplantation, to further prevent or delay the immunopathology.

In this regard, a particular object of this invention is a method of treating (including, without limitation, preventing, reducing, alleviating or delaying) GVHD in a subject undergoing HSC transplantation, the method comprising administering to the subject, prior to, during or after HSC transplantation, an amount of immunoregulatory T cells effective at treating GVHD in said subject. In preferred embodiments, the cells are autologous, freshly isolated or ex vivo expanded, and/or genetically modified to encode a conditionally toxic molecule, and/or administered together with transplantation, optionally followed by subsequent administration(s) depending on the appearance of delayed clinical signs of GVHD. The method if particularly suited for treating GVHD associated with Bone Marrow Transplantation.

An other particular object of this invention resides in a composition comprising (i) freshly isolated or ex vivo expanded human immunoregulatory T cells and (ii) donor effector T cells, for combined, separate or sequential use. The composition may further comprise (iii) hematopoietic stem cells. Such compositions are particularly suited for conducting bone marrow transplantation.

An other particular object of this invention is a method of treating (including, without limitation, preventing, reducing, alleviating or delaying) organ transplant rejection in a subject undergoing organ transplantation, the method comprising administering to the subject, prior to, during or after organ transplantation, an amount of immunoregulatory T cells effective at reducing organ rejection in said subject. In preferred embodiments, the cells are autologous, freshly isolated or ex vivo expanded, and/or genetically modified, and/or administered together with transplantation, optionally followed by subsequent administration(s) depending on the appearance of delayed clinical signs of organ rejection.

An other particular object of this invention is a method of treating (including, without limitation, preventing, reducing, alleviating or delaying) an autoimmune disease in a subject, the method comprising administering to the subject, an amount of immunoregulatory T cells effective at reducing said autoimmune disease in said subject, particularly an amount effective at suppressing the activity of effector T cells responsible for said autoimmune disease. In preferred embodiments, the cells are autologous, ex vivo expanded in the presence of an auto-antigen involved in said auto-immune disease, and/or genetically modified. Particularly useful immunoregulatory T cells are those which are genetically modified to express a recombinant T cell receptor (or a sub-unit or fragment thereof) specific for an autoantigen.

An other particular object of this invention is a method of treating (including, without limitation, preventing, reducing or alleviating) allergy in a subject, the method comprising administering to the subject, an amount of immunoregulatory T cells effective at reducing said allergy in said subject, particularly an amount effective at suppressing the activity of effector T cells responsible for said allergy. In preferred embodiments, the cells are autologous, ex vivo expanded in the presence of an allergen involved in said auto-immune disease, and/or genetically modified.

Further aspects and advantages of the present invention will be disclosed in the following experimental section, which should be regarded as illustrative and non limiting. All references cited are incorporated therein by reference.

### LEGEND TO THE FIGURES

FIG. 1. CD4⁺CD25⁺ regulatory T-cells naturally present in the transplant modulate GVHD. Lethally irradiated [B6×D2]F1 mice were grafted with semi-allogeneic B6 BM cells supplemented with either 10×10⁶ total B6 T-cells (white circle, n=10), or 10×10⁶ CD25-depleted B6 T-cells (black circle, n=10). Cumulative results from two independent experiments are shown. Kaplan-Meier survival curves were established for each group with P-values indicated.

FIG. 2. Prevention of GVHD by the addition of fresh CD4⁺CD25⁺ regulatory T-cells. Lethally irradiated mice were grafted with allogeneic BM cells supplemented with either 10×10⁶ T-cells (white circles, n=5) or 10×10⁶ T-cells and 5×10⁶ freshly isolated CD4⁺CD25⁺ T-cells (black triangles, n=4). (A) Survival of [B6×D2]F1 recipients transplanted with semi-allogeneic B6 cells. (B) Survival of C3H recipients transplanted with fully allogeneic BALB/c cells. Kaplan-Meier survival curves were shown with P-values indicated.

FIG. 3. Ex vivo expanded CD4⁺CD25⁺ T-cells maintain their immuno-regulatory properties in GVHD. (A) 0.2×10⁶ B6 (white circle) or 5.5×10⁶ BALB/c (black circle) purified CD4⁺CD25⁺CD62L^{high} T-cells were stimulated with IL-2 and irradiated splenocytes from [B6×D2] or C3H mice, respectively. The graph depicts expansion of living cells. (B) Flow cytometry analyses for expression of CD4, CD25 and CD62L (inset) on total cells and on CD4⁺CD25⁺CD62L^{hogh} T-cells after cell sorting (fresh) and after 2 wk of stimulation with allogeneic irradiated splenocytes and IL-2 (cultured). (C) CD4⁺CD25⁺CD62L^{high} T-cells from BALB/c mice were stimulated with C3H APCs (left panel) or B6 APCs (right panel). After 2 week of culture, T-cells were restimulated with either the same allogeneic APCs (black circle) or third party allogeneic APCs (white circle; B6 in the left panel and C3H in the right panel). Proliferation was assessed after 2, 2.5 or 3 d of stimulation. In both assays, T-cell proliferation to third party allogeneic APCs and the one obtained in the culture without APCs were comparable and below 10,000 cpm.

FIG. 4. Prevention of GVHD by the addition of expanded CD4⁺CD25⁺ regulatory T cells. At the end of the culture (day 15 and 28 for regulatory T cells from BALB/c and B6 mice, respectively), expanded regulatory T cells were tested for their capacity to control GVHD. Lethally irradiated mice were grafted with allogeneic BM cells supplemented with either 10×10⁶ fresh T cells (white circles, n=5 per group) or 10×10⁶ fresh T cells and 7×10⁶ expanded CD4⁺CD25⁺ T cells (black triangles, n=5 per group). For both genetic combinations, the addition of expanded CD4⁺CD25⁺ T cells statistically increased the survival of mice. Kaplan-Meier survival curves were shown with P-values indicated.

### EXAMPLES

### Materials and Methods

HSCT.

C57B1/6 (B6), (H-2^{b}), BALB/c (H-2^{d}), and [B6×DBA/2(D2)]F1 (H-2^{bxd}) and C3H (H-2^{k}) mice were obtained from Iffa Credo (L'Arbresle, France). Mice were manipulated according to European Economic Community guidelines. Experiments were performed as described (23), except otherwise stated. Briefly, 24 hours after lethal irradiation of [B6×D2]F1 (11 Gy) or C3H (9.5 Gy) mice, recipients were transplanted with cells from B6 or BALB/c donor mice, respectively. The transplants were constituted of 5×10⁶ T-depleted bone marrow (BM) cells, 10×10⁶ T-cells collected from pooled spleen and peripheral LN (referred to as total T-cells in the text) and, when indicated, purified of CD4⁺CD25⁺ T-cells. In control mice, the transplantation of only the T-depleted BM cells did not induce GVHD.

Purification of CD4⁺CD25⁺ T-cells.

Cells from spleen and peripheral LN were sequentially incubated with saturating amounts of biotin-labeled anti-CD25 antibody (7D4, PharMingen, San Diego, Calif.) and streptavidin microbeads (Miltenyi Biotec, Bergisch Gladbach, Germany) for 30 min on ice, followed by purification of magnetic cell separation using LS columns (Miltenyi Biotec), according to the protocol advised by Miltenyi Biotec. To increase cell purification, the cells of the positive fraction were separated on another LS column. All steps were performed in phosphate buffer saline with 3% serum. The purity of CD4⁺CD25⁺ T-cells was of 80-85%. The CD25-depleted cells which did not bind to the anti-CD25-coated beads were harvested from the flow through and contained less than 0.3% of CD4⁺ CD25hu + T-cells. The fresh CD4⁺CD25⁺ T-cells and the CD25-depleted cells were washed twice with PBS before injection in HSCT. For in vivo cell expansion, CD4⁺CD25⁺ T-cells were further enriched. Cells were stained for 30 min on ice with FITC-labeled anti-CD4 (GK1.5, PharMingen), phycoerythrine-labeled anti-CD62L (MEL-14, PharMingen) and streptavidin-Cy-Chrome (PharMingen) which bound to free biotin-labeled CD25 molecules, uncoupled to beads. The CD4⁺CD25⁺CD62L^{high} T-cells were sorted on a FACstar+(Becton Dickinson, San Jose, Calif.), giving a purity of 99%.

Culture of CD4⁺CD25⁺CD62L^{high} T Cells.

Highly purified CD4⁺CD25⁺CD62L^{high} T-cells from B6 or BALB/c mice were stimulated with total splenocytes from [B6×D2]F1 or C3H mice, respectively. Cultures were performed in RPMI 1640 (Gibco BRL, Life Technologies, Paisley, UK) supplemented with 10% FCS (Gibco BRL), L-glutamine, antibiotics, 10 mM BEPES, 5×10⁻⁵ 2-β-mercaptoethanol and 30 ng/ml of mouse IL-2 (R & D system, Oxon, UK). At the beginning, 1×10⁶ CD4⁺CD25⁺CD62L^{high} T-cells per ml were co-cultured with 2×10⁶ irradiated (20 Gy) splenocytes per ml. After 5 d of culture, cells were counted and cell density adjusted to 1×10⁶ per ml with fresh medium if necessary. At day 8, cells were re-seeded at 0.1×10⁶ per ml and re-stimulated with 2×10⁶ irradiated splenocytes per ml. After 4 d, cells were counted and cell density adjusted to 0.2×10⁶ per ml with fresh medium if necessary. Following cycles of stimulation were similarly performed. Cells were analyzed by flow cytometry after staining with FITC-labeled anti-CD4 (GK1.5, PharMingen), phycoerythrine-labeled anti-CD62L (MEL-14, PharMingen) and streptavidin-Cy-Chrome (PharMingen) on a FACSCalibur (Becton Dickinson, San Jose, Calif.) or washed twice in phosphate buffer saline and used for HSCT.

Proliferation Assays.

CD4⁺CD25⁺CD62L^{high} purified from BALB/c mice were stimulated for 15 d by irradiated C3H or B6 splenocytes as described previously. Then, 1×10⁵ T cells of both cultures were restimulated by either 1×10⁶ irradiated C3H or B6 splenocytes in the presence of IL-2 (30 ng/ml) for 48-72h and were pulsed with [³H] methyl-thymidine for the last 15h.

Statistical Analyses.

Statistical analyses were performed using Statview software (SAS Inc.). Kaplan-Meier survival curves were established for each group. P-values for Logrank test are indicated.

### Results and Discussion

CD4⁺CD25⁺ Regulatory T-cells Modulate GVHD

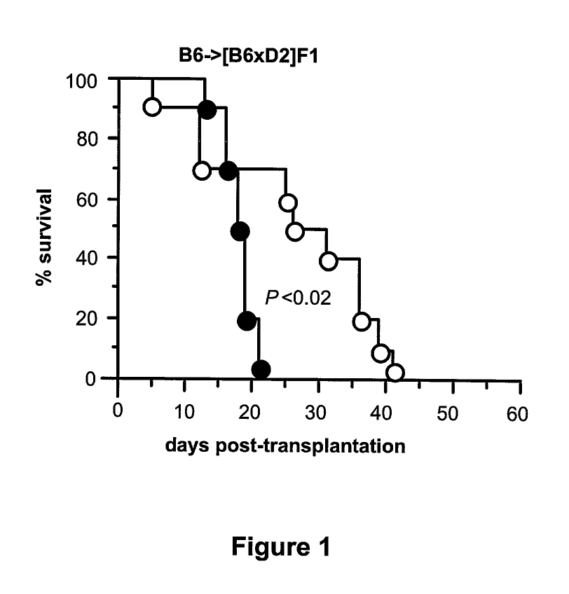

CD4⁺CD25⁺ T cells represent 5-10% of the normal T-cell compartment in mice and humans (7, 24). During allogeneic HSCT, donor T-cells are present in the transplant. Consequently, when grafted, patients also receive CD4⁺CD25⁺ regulatory T-cells. We first analyzed if this population plays a role in the control of GVHJD. In our murine model, CD4⁺CD25⁺ T cells represent 3-5% of the donor cells collected from spleen and LN. The incidence of GVHD was compared after allogeneic HSCT of lethally irradiated [C57BL/6 (B6)×DBA/2 (D2)]F1 mice receiving BM cells together with either total donor T-cells or CD25-depleted donor T-cells from B6 mice. In this semi-allogeneic combination between donor and recipient, the infusion of 10×10⁶ total T-cells induced lethal GVHD (FIG. 1). All mice had ongoing clinical signs of GVHD and were dead by day 41. When mice were grafted with the same number of CD25-depleted T-cells, the onset of clinical signs of GVHD such as weight loss, diarrhea and hunching, appeared much faster and all mice were dead by day 21 post-transplantation (FIG. 1). This result revealed an unforeseen effect of CD4⁺CD25⁺ regulatory T-cells present in the transplant, i.e. that they play a major role in the control of GVHD.

Prevention of GVHD by Addition of Fresh Immunoregulatory T Cells

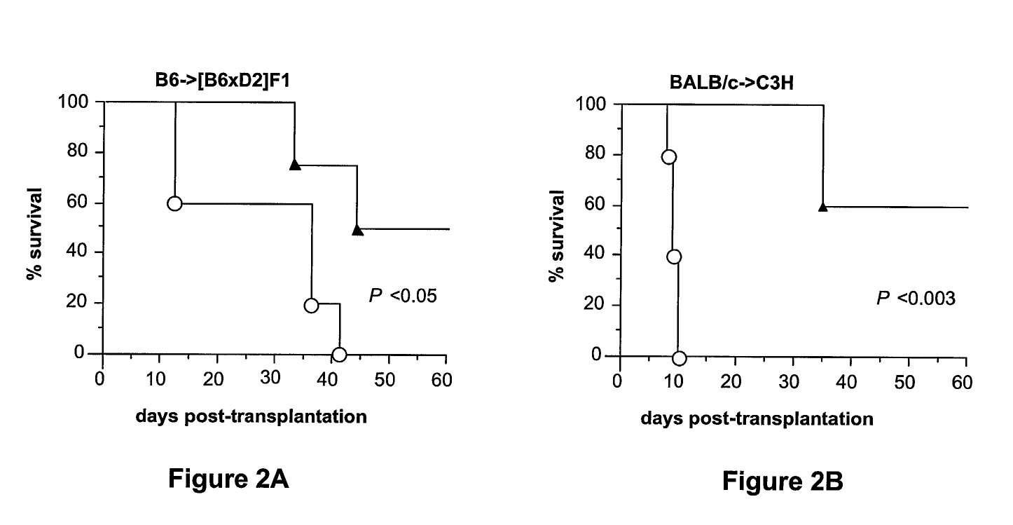

The effect of regulatory T-cells on GVHD following HSCT suggested their potential use for therapeutic intervention. We thus investigated whether GVHD would be delayed if additional numbers of CD4⁺CD25⁺ T-cells were injected. First, we verified that CD4⁺CD25⁺ T-cells themselves did not induce GVHD. When lethally irradiated mice were grafted with a BM transplant supplemented with 5×10⁶ CD4⁺CD25⁺ purified T-cells, no GVHD was observed (data not shown). We then grafted irradiated [B6×D2]F1 mice with BM cells and 10×10⁶ total T-cells supplemented with 5×10⁶ CD4⁺CD25⁺ purified T-cells from B6 mice. These mice remained healthy until about day 25, as opposed to the control mice (BM cells plus total T-cells) which rapidly developed clinical signs of GVHD from day 8-10 (data not shown). Significantly, 2 out of 4 mice receiving additional regulatory T-cells survived without any further treatment (FIG. 2A). When these two mice were sacrificed at day 60, we did not observe any histopathological signs of GVHD in the liver, a target organ of GVHD, and one mouse displayed moderate signs of GVHD in the spleen (data not shown). We reproduced this experiment in a different genetic combination. When C3H mice were grafted with BALB/c donor cells, GVHD-related mortality was very fast in the control group transferred with BM cells and 10×10⁶ total T-cells (100% of mice died by day 10). The addition of 5×10⁶ CD4⁺CD25⁺ purified T-cells significantly delayed mortality as compared to the control group. Clinical signs of GVHD were not observed before day 29 and no mice died until day 35 (FIG. 2B). At day 60, 3 out of 5 mice did not display any clinical signs of GVHD. Altogether, these results demonstrate that the sole addition of fresh CD4⁺CD25⁺ regulatory T-cells significantly delays or even prevents GVHD after allogeneic HSCT.

Ex vivo Expanded Immunoregulatory Cells Control GVHD

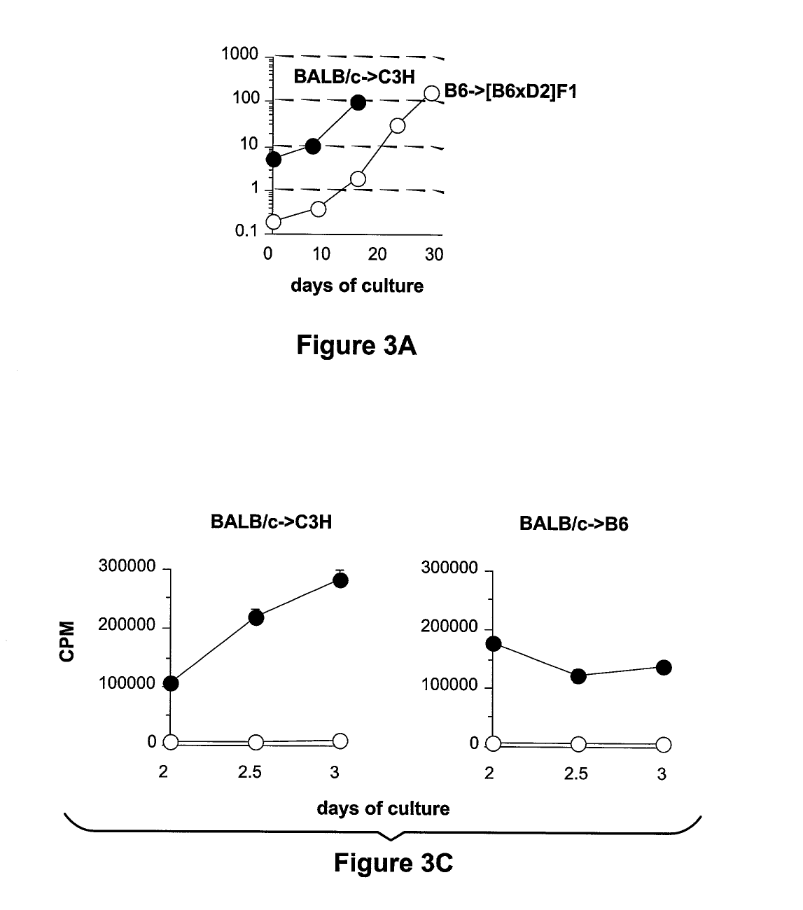

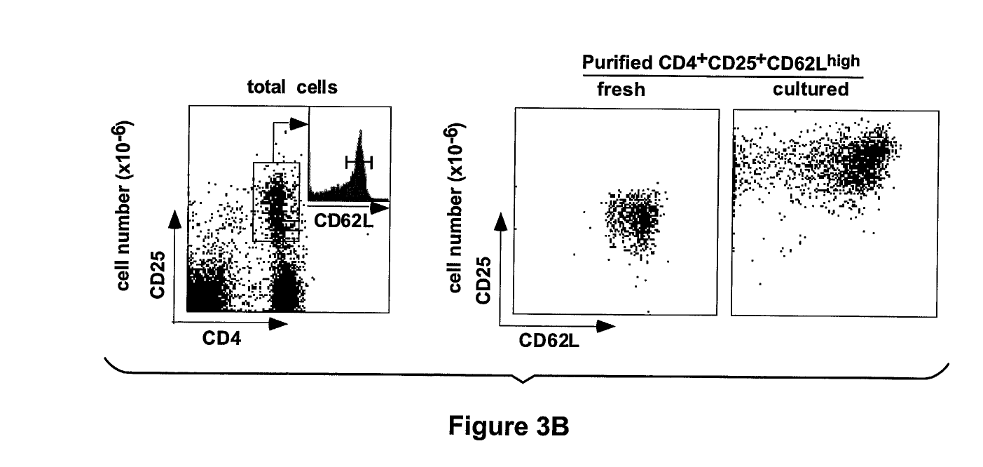

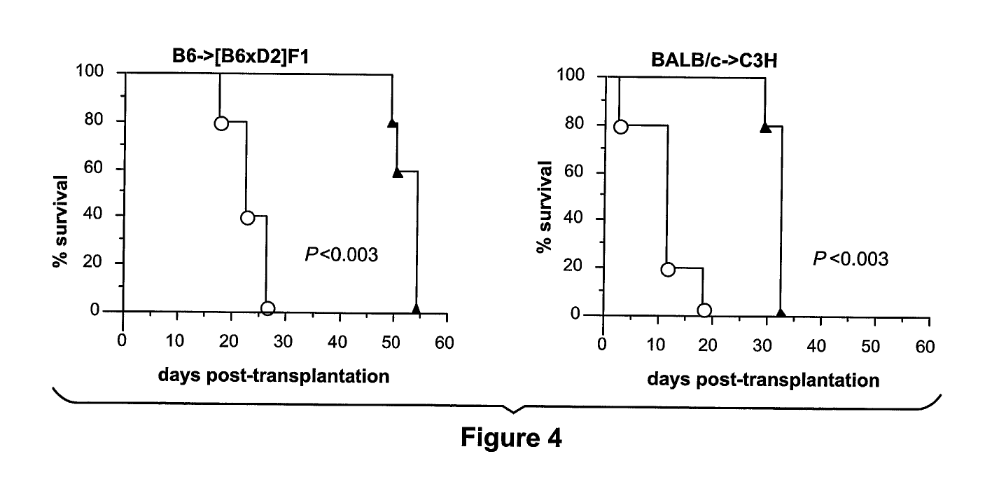

A potential limitation in the utilization of regulatory T-cells for preventing GVHD is the difficulty to obtain a sufficient number of these relatively rare cells. We thus tested whether they could be expanded while retaining their functional properties. We chose to stimulate these cells by allogeneic antigen presenting cells in the presence of IL-2 with the aim to increase their number (24-27) and their specificity to recipient-type alloantigens. For this, we started with highly purified populations of CD4⁺CD25⁺CD62L^{high} T-cells constituting the major fraction of the CD4⁺CD25⁺ regulatory T-cells, in order to limit the contamination with conventional activated CD4⁺CD25⁺CD62L^{low} T-cells. Then, the cells purified from BALB/c or B6 mice were co-cultured with irradiated C3H or [B6×D2]F1 splenocytes, respectively. In both cultures, regulatory T-cells rapidy expanded. From 5.5×10⁶ BALB/c CD4⁺CD25⁺ T-cells, we were able to produce 100×10⁶ regulatory T-cells (20-fold expansion) after 15 d of culture. In the same manner, the number of B6 CD4⁺CD25⁺ T-cells was increased 10-fold during the first 2 wk and 100-fold during the following 2 wk of culture (FIG. 3A). Similar expansion was observed in another genetic combination, when BALB/c CD4⁺CD25⁺ T-cells were stimulated by B6 splenocytes (data not shown). Importantly, these cells kept the phenotype of regulatory T-cells since they expressed even higher levels of CD25 and they maintained high levels of CD62L expression (FIG. 3B). Since regulatory T cells were cultured in presence of allogeneic splenocytes, we tested whether expanded regulatory T cells were specific to these alloantigens. After 2 wk of culture of BALB/c regulatory T cells stimulated by irradiated C3H APCs, these cells did not respond to B6 APCs whereas they continue to proliferate to C3H APCs. Similar findings were observed when using B6 APCs instead of C3H APCs (FIG. 3C).

To test the capacity of the ex vivo expanded CD4⁺CD25⁺CD62L^{high} T-cells to regulate GVHD, we performed experiments similar to those presented in FIG. 2 using cultured CD4⁺CD25⁺CD62L^{high} T-cells instead of freshly isolated CD4⁺CD25⁺ T-cells. In the B6→[B6×D2]F1 combination, the addition of 7×10⁶ CD4⁺CD25⁺ expanded T-cells to BM cells and 10×10⁶ total T-cells remarkably prolonged the survival as compared to the control group (FIG. 3C). This observation was confirmed in the BALB/c-→C3H combination. Remarkably, in both genetic combinations, the mice which had received cultured CD4⁺CD25⁺ regulatory T-cells appeared completely healthy for several wk.

In sum, these results demonstrate that a high number of CD4⁺CD25⁺ T-cells can be generated ex vivo without altering their phenotype nor their regulatory property toward GVHD. The results also demonstrate that ex vivo expanded CD4⁺CD25⁺ T-cells have a strong impact on the survival of grafted mice. Sequential injection(s) of these cells could induce further long term protection from GVHD.

Discussion

The present invention thus shows, for the first time, that the few regulatory T-cells naturally present in the inoculum during allogeneic HSCT significantly delays the occurrence of GVHD and the linked mortality. Recently, strategies of ex vivo depletion of alloreactive effector T-cells before HSCT have been proposed to modulate GVHD [Cavazzana-Calvo, 1994 #26; Fehse, 2000 #29]. In these reports, CD25⁺ cells were depleted after in vitro stimulation of donor T cells by recipient cells. In such a procedure, not only alloreactive effector T-cells but also the population of regulatory T-cells will be depleted, thus challenging the expected therapeutic effect.

It has been suggested that CD4⁺CD25⁺ regulatory T-cells may regulate autoimmune diseases [Sakaguchi, 1995 #19; Salomon, 2000 #20] and rejection of allogeneic solid organ transplantation [Hara, 2001 #9; Gregori, 2001 #7]. Here, we now show that this cell population regulates GVHD and can be used in cell therapy. A therapeutic effect of these cells for the prevention of autoimmune diseases had only been suggested to date in CD25-deficient animals [Sakaguchi, 1995 #19; Suri-Payer, 1998 #34; Salomon, 2000 #20]. Taylor et al. showed that CD4⁺CD25⁺ T-cells had a modest capacity to down modulate activation of alloreactive specific CD4⁺ T cells in vivo [Taylor, 2001 #11]. Finally, CD4⁺CD25⁺ regulatory T-cells have been demonstrated to efficiently prevent rejection of allogeneic solid organ transplants, but this effect was obtained with cells purified from mice which had previously received a treatment for tolerance induction [Hara, 2001 #9; Gregori, 2001 #7]. The present invention provides the first report demonstrating that freshly isolated CD4⁺CD25⁺ regulatory T-cells from unmanipulated animals can be used in cell therapy of an immunopathology.

In GVHD, we obtained an improved therapeutic effect after the addition of regulatory T-cells in about the same proportion than total donor T-cells. In human HSCT, an order of 3 billions T-cells are usually present in the infused transplant [Blaise, 2000 #39], whereas a maximum of 100 millions CD4⁺CD25⁺ regulatory T-cells can be collected from the blood of the same donor. This led us to test the functionality of CD4⁺CD25⁺ regulatory T-cells in GVHD, after their ex vivo expansion. Previous publications demonstrated that cultured regulatory T-cells from both mice and humans remained functional but their suppressor activity was only attested by in vitro assays [Takahashi, 1998 #37; Thornton, 2000 #21; Levings, 2001 #36; Jonuleit, 2001 #30]. Here, we show for the first time that extensively expanded regulatory T-cells can still be used to control an immunopathological process in vivo and, consequently, can be envisaged as a new therapeutic tool when a large number of regulatory T-cells is required. The ex vivo expansion procedure of regulatory T-cells stimulated by recipient-type alloantigens presents three additional advantages. First, the repertoire of regulatory T-cells specific to recipient alloantigens should be selected while non-alloreactive cells would die during the culture in the absence of TCR-mediated activation, as suggested from a previous work [Jonuleit, 2001 #30]. In this case, the regulatory effects of these expanded cells would be preferentially targeted to the pathogenic donor T cells specific to the recipient alloantigens. Consequently, GVHD would be controlled without altering the immune reconstitution after allogeneic HSCT. Second, the ex vivo expansion of regulatory T-cells is compatible with retroviral gene transfer. This clearly represents a benefit if the selective elimination of these cells through a suicide gene is envisaged, a strategy previously reported for effector T-cells[Cohen, 1999 #25]. Finally, in our culture conditions, the capacity to produce high numbers of regulatory T-cells should allow to improve this therapeutic approach. In our experimental model, mice receiving a single injection of expanded regulatory T-cells at time of HSCT are free of any signs of GVBD for several weeks. Then, to circumvent any reduced survival of ex vivo expanded regulatory T-cells administered in vivo, sequential injection of CD4⁺CD25⁺ regulatory T-cells may be performed, if necessary.

To date, standard preventive treatments of GVHD are only partially efficient since 15 to 60% of the patients indeed develop GVHD [Storb, 1989 #32; Socie, 1998 #33; Ratanatharathom, 1998 #31]. Similarly, we observed that in mice, cyclosporin A which is part of these standard treatments, had no effect on GVKD (Maury et al. submitted). Using the same experimental conditions of allogeneic HSCT, we now demonstrate that the use of CD4⁺CD25⁺ regulatory T-cells dramatically improve the prevention of GVHD. This leads us to propose CD4⁺CD25⁺ regulatory T-cells as new therapeutic tool for controlling graft-versus-host disease in allogeneic hematopoietic stem cell transplantation or other immunopathology.

### References

1. Thomas, E. D., R. Storb, R. A. Clift, A. Fefer, L. Johnson, P. E. Neiman, K. G. Lemer, H. Glucksberg, and C. D. Buckner. 1975. Bone-marrow transplantation. N Engl J Med 292:895-902.

2. Martin, P. J., J. A. Hansen, C. D. Buckner, J. E. Sanders, H. J. Deeg, P. Stewart, F. R. Appelbaum, R. Clift, A. Fefer, R. P. Witherspoon, and et al. 1985. Effects of in vitro depletion of T cells in HLA-identical allogeneic marrow grafts. Blood 66:664-72.

3. Mackall, C. L., and R. E. Gress. 1997. Thymic aging and T-cell regeneration. Immunol Rev 160:91-102.

4. Horowitz, M. M., R. P. Gale, P. M. Sondel, J. M. Goldman, J. Kersey, H. J. Kolb, A. A. Rimm, O. Ringden, C. Rozman, B. Speck, and et al. 1990. Graft-versus-leukemia reactions after bone marrow transplantation. Blood 75:555-62.

5. Storb, R., H. J. Deeg, M. Pepe, F. Appelbaum, C. Anasetti, P. Beatty, W. Bensinger, R. Berenson, C. D. Buckner, R. Clift, and et al. 1989. Methotrexate and cyclosporine versus cyclosporine alone for prophylaxis of graft-versus-host disease in patients given HLA-identical marrow grafts for leukemia: long-term follow-up of a controlled trial. Blood 73:1729-34.

6. Socie, G., and J. Y. Cahn. 1998. Acute graft-versus-host disease. The clinical practice of stem-cell transplantation Isis Medical Media Ltd.: Oxford,: 595-618.

7. Sakaguchi, S., N. Sakaguchi, M. Asano, M. Itoh, and M. Toda. 1995. Immunologic self-tolerance maintained by activated T cells expressing IL-2 receptor alpha-chains (CD25). Breakdown of a single mechanism of self-tolerance causes various autoimmune diseases. J Immunol 155:1151-64.

8. Salomon, B., D. J. Lenschow, L. Rhee, N. Ashourian, B. Singh, A. Sharpe, and J. A. Bluestone. 2000. B7/CD28 costimulation is essential for the homeostasis of the CD4+CD25+ immunoregulatory T cells that control autoimmune diabetes. Immunity 12:431-40.

9. Suri-Payer, E., A. Z. Amar, A. M. Thornton, and E. M. Shevach. 1998. CD4+CD25+ T cells inhibit both the induction and effector function of autoreactive T cells and represent a unique lineage of immunoregulatory cells. J Immunol 160:1212-8.

10. Stephens, L. A., and D. Mason. 2000. CD25 is a marker for CD4+ thymocytes that prevent autoimmune diabetes in rats, but peripheral T cells with this function are found in both CD25+ and CD25− subpopulations. J Immunol 165:3105-10.

11. Sakaguchi, S. 2000. Regulatory T cells: key controllers of immunologic self-tolerance. Cell 101:455-8.

12. Chatenoud, L., B. Salomon, and J. A. Bluestone. 2001. Suppressor T cells—they're back and critical for regulation of autoimmunity! Immunol Rev 182:149-63.

13. Shevach, E. M., R. S. McHugh, C. A. Piccirillo, and A. M. Thornton. 2001. Control of T-cell activation by CD4+CD25+ suppressor T cells. Immunol Rev 182:58-67.

14. Cederbom, L., H. Hall, and F. Ivars. 2000. CD4+CD25+ regulatory T cells down-regulate co-stimulatory molecules on antigen-presenting cells. Eur J Immunol 30:1538-43.

15. Takahashi, T., T. Tagami, S. Yamazaki, T. Uede, J. Shimizu, N. Sakaguchi, T. W. Mak, and S. Sakaguchi. 2000. Immunologic self-tolerance maintained by CD25(+)CD4(+) regulatory T cells constitutively expressing cytotoxic T lymphocyte-associated antigen 4. J Exp Med 192:303-10.

16. Nakamura, K., A. Kitani, and W. Strober. 2001. Cell contact-dependent immunosuppression by CD4(+)CD25(+) regulatory T cells is mediated by cell surface-bound transforming growth factor beta. J Exp Med 194:629-44.

17. Powrie, F., J. Carlino, M. W. Leach, S. Mauze, and R. L. Coffman. 1996. A critical role for transforming growth factor-beta but not interleukin 4 in the suppression of T helper type 1-mediated colitis by CD45RB(low) CD4+ T cells. J Exp Med 183:2669-74.

18. Asseman, C., S. Mauze, M. W. Leach, R. L. Coffman, and F. Powrie. 1999. An essential role for interleukin 10 in the function of regulatory T cells that inhibit intestinal inflammation. J Exp Med 190:995-1004.

19. Seddon, B., and D. Mason. 1999. Regulatory T cells in the control of autoimmunity: the essential role of transforming growth factor beta and interleukin 4 in the prevention of autoimmune thyroiditis in rats by peripheral CD4(+)CD45RC- cells and CD4(+)CD8(−) thymocytes. J Exp Med 189:279-88.

20. Taylor, P. A., R. J. Noelle, and B. R. Blazar. 2001. CD4(+)CD25(+) immune regulatory cells are required for induction of tolerance to alloantigen via costimulatory blockade. J Exp Med 193:1311-8.

21. Hara, M., C. I. Kingsley, M. Niimi, S. Read, S. E. Turvey, A. R. Bushell, P. J. Morris, F. Powrie, and K. J. Wood. 2001. IL-10 is required for regulatory T cells to mediate tolerance to alloantigens in vivo. J Immunol 166:3789-96.

22. Gregori, S., M. Casorati, S. Amuchastegui, S. Smiroldo, A. M. Davalli, and L. Adorini. 2001. Regulatory t cells induced by 1alpha,25-dihydroxyvitamin d(3) and mycophenolate mofetil treatment mediate transplantation tolerance. J Immunol 167:1945-53.

23. Cohen, J. L., S. Lacroix-Desmazes, F. Charlotte, L. Lejeune, P. J. Martin, D. Klatzmann, and O. Boyer. 1999. Immunological defects after suicide gene therapy of experimental graft-versus-host disease. Hum Gene Ther 10:2701-7.

24. Levings, M. K., R. Sangregorio, and M. G. Roncarolo. 2001. Human cd25(+)cd4(+) t regulatory cells suppress naive and memory T cell proliferation and can be expanded in vitro without loss of function. J Exp Med 193:1295-302.

25. Takahashi, T., Y. Kuniyasu, M. Toda, N. Sakaguchi, M. Itoh, M. Iwata, J. Shimizu, and S. Sakaguchi. 1998. Immunologic self-tolerance maintained by CD25+CD4+ naturally anergic and suppressive T cells: induction of autoimmune disease by breaking their anergic/suppressive state. Int Immunol 10: 1969-80.

26. Thornton, A. M., and E. M. Shevach. 2000. Suppressor effector function of CD4+CD25+ immunoregulatory T cells is antigen nonspecific. J Immunol 164:183-90.

27. Jonuleit, H., E. Schmitt, M. Stassen, A. Tuettenberg, J. Knop, and A. H. Enk. 2001. Identification and functional characterization of human CD4(+)CD25(+) T cells with regulatory properties isolated from peripheral blood. J Exp Med 193:1285-94.

28. Jung, T. M., W. M. Gallatin, I. L. Weissman, and M. O. Dailey. 1988. Down-regulation of homing receptors after T cell activation. J Immunol 141:4110-7.

29. Cohen, J. L., O. Boyer, and D. Klatzmann. 1999. Would suicide gene therapy solve the ‘T-cell dilemma’ of allogeneic bone marrow transplantation? Immunol Today 20:172-6.

1. A method of treatment of an immune disease in a subject, comprising administering to a subject in need thereof an amount of immunoregulatory T cells effective at suppressing a pathological immune response.

2. The method of claim 1, wherein the immunoregulatory T cells are autologous or allogeneic with respect to the subject to be treated.

3. The method of claim 1 or 2, wherein the immunoregulatory T cells are freshly isolated from a biological fluid.

4. The method of claim 1 or 2, wherein the immunoregulatory T cells are expanded ex vivo or in vitro.

5. The method of claim 1 or 2, wherein the immunoregulatory T cells are genetically modified.

6. The method of claim 1, wherein between 10E5 to 10E10 immunoregulatory T cells are administered.

7. The method of claim 1, wherein the immunoregulatory T cells are obtained by a method comprising:
a) providing a biological sample comprising lymphocytes,
b) isolating immunoregulatory T cells from said sample,
c) optionally expanding the immunoregulatory T cells by activation in the presence of a stimulating agent and a cytokine,
d) optionally genetically modifying the immunoregulatory T cells by contacting said cells with a recombinant nucleic acid molecule, and
e) conditioning said cells in the presence of a pharmaceutically acceptable medium or vehicle.

8. The method of claim 1, for the treatment of a disease caused by pathological T cells.

9. The method of claim 1, for the treatment of graft versus host disease in a subject undergoing allogeneic organ transplantation.

10. The method of claim 1, for the treatment of disease selected from an autoimmune disease, allergy, organ transplant rejection and viro-induced immunopathology.

11. The method of claim 1, wherein the treatment is preventive.

12. The method of claim 1, wherein the treatment is curative.

13. The method of claim 1, for the treatment of graft versus host disease in a subject undergoing allogeneic bone marrow transplantation, comprising administering to the subject an amount of freshly isolated or ex vivo expanded human immunoregulatory T cells effective at suppressing or reducing the activity of effector T cells responsible for graft versus host disease in the subject.

14. The method of claim 13, wherein the immunoregulatory T cells are allogeneic.

15. The method of claim 13, wherein the immunoregulatory T cells are genetically modified and comprise a recombinant nucleic acid molecule encoding a product with conditional toxicity to said cells.

16. The method of claim 13, wherein the immunoregulatory T cells are administered to the subject together with the bone marrow transplant or after the bone marrow transplant.

17. A composition comprising genetically modified freshly isolated or ex vivo expanded human immunoregulatory T cells and a pharmaceutically acceptable medium or vehicle.

18. A method of producing human immunoregulatory T cells, comprising:
a) providing a biological sample comprising lymphocytes,
b) isolating immunoregulatory T cells from said sample,
c) expanding the immunoregulatory T cells by activation in the presence of a stimulating agent and a cytokine, and
d) optionally genetically modifying the immunoregulatory T cells by contacting said cells with a recombinant nucleic acid molecule.
